(19) United States
(12) Patent Application Publication
(10) Pub. No.: US 2008/0097363 A1
(43) Pub. Date: Apr. 24, 2008

(54) Pant type absorbent article

(75) Inventors: Ingemar Fernfors, Molndal (SE); Birgitta Karlsson, Molnlycke (SE)

Correspondence Address: BUCHANAN, INGERSOLL & ROONEY PC, POST OFFICE BOX 1404, ALEXANDRIA, VA, 22313-1404, US

(73) Assignee: SCA HYGIENE PRODUCTS AB, Goteborg (SE)

(21) Appl. No.: 11/819,739

(22) Filed: Jun. 28, 2007

Related U.S. Application Data
(63) Continuation of application No. PCT/SE04/02026, filed on Dec. 29, 2004.

(51) Int. Cl. A61F 13/15

(52) U.S. Cl. 604/365; 604/385.03; 604/387; 604/396; 604/385.01

(57) ABSTRACT

A pant type absorbent article having a front region (2), a back region (3) and a crotch region (4) between the front region and back region, the front and back regions being joined to each other along two opposite side edges (2a, 3a) of the article so as to form side joint regions (10). The article further includes at least one fastener (12) joined to at least one of the front and back regions for allowing the article to be secured in a configuration that provides a convenient disposal after the article has been used and/or a reclosure of the article if opened during use. The fastener (12) is disposed in the side joint region (10) and is hidden between the joined side edges of the back and front regions during normal use of the article, and is exposed upon tearing open at least a part of the side joint region.

### CROSS-REFERENCE TO RELATED APPLICATIONS

The present application is a continuation of PCT/SE2004002026, filed on Dec. 29, 2004, and which designates the U.S. The entire contents of PCT/SE2004/002026 are incorporated herein by reference.

### TECHNICAL FIELD

The present invention refers to a pant type absorbent article such as a pant diaper, a sanitary pant or incontinence pant.

### BACKGROUND

Pant type absorbent articles like pant diapers, sanitary pants and incontinence pants are capable of being pulled up over the hips of the wearer to allow the wearer or caregiver to easily put on the article. These pant diapers have side joint regions joining the front and back regions together by gluing, ultrasonic welding or the like. In order to fit about the waist and legs of the wearer without sagging or sliding down and to allow the pant diaper to be easily pulled up over the hips of the wearer, at least part of the waist area, the leg opening area and the side areas adjacent the side joint regions are elasticized. The elastification is usually accomplished by elastic members, such as elastic threads, contractably affixed between the backsheet and the topsheet. It is further known to make portions of the chassis of absorbent articles of an elastic material, such as stretch-bonded laminates.

After the pant diaper is soiled it is usually removed from the wearer by tearing open the side joint regions. The removed pant diaper is then folded or rolled up so that the soiled portion is wrapped inside for disposal. In order to prevent the soiled pant diaper from unfolding and to keep the soiled portion inside, it is desired that a so called disposal means, which is a fastening means such as a tape tab or a hook member, is arranged to keep the diaper in the folded or rolled up state. Usually the disposal means is a strip of fastening means, for example adhesive or mechanical fastening means, and that has one end fixed to the diaper on the front or rear waist region and is folded up, so that in normal use the adhesive or the mechanical fastening means is brought in an unexposed position. The strip may be unfolded so as to expose the fastening means when it is desired to use it as a disposal means.

EP-A-1 121 917 discloses a pant diaper having a disposal means in the form of tape strips arranged in vicinity of the side edges of the diaper. Each tape strip comprises two portions, a first portion extending longitudinally in parallel to the side edges and a second portion extending from a predetermined region of the first portion longitudinally or transversely of the diaper. The disposal means is told to be adapted to hold the diaper in its rolled up state for disposal without leakage through the waist- or leg-openings.

EP-A-1 121 918 also discloses a pant diaper having a disposal means in the form of tape strips arranged in vicinity of the side edges of the diaper. Each tape strip extends longitudinally in parallel to the respective side edge and has its opposite ends bonded to the diaper in vicinity to the waist-opening and the leg-opening respectively. The intermediate region of the tape strip has an adhesive region adapted to be separably bonded to the outer peripheral surface of the diaper rolled up for disposal. The disposal means is told to be adapted to hold the diaper in its rolled up state for disposal without leakage through the waist- or leg-openings.

WO 98/53780 discloses a pant diaper having elastically stretchable side panels which are joined to each other in an overlapped manner. A tear open tab extends laterally outward from the side panel and a disposal means is provided on the tear open tab.

All these previously known disposal means may be opened and even by mistake be removed by the user or caregiver before the pant diaper is removed for disposal. The disposal means may further be opened due to movements between the diaper and the clothing and can then stick to the clothing.

### OBJECT AND SUMMARY

One object is to provide a pant type absorbent article having a disposal means that may not by mistake be opened before the diaper is ready for disposal after use. There is provided a pant type absorbent article of the type mentioned above and in which the fastening means used for disposal of the article is disposed in the side joint regions and is hidden between the joined side edges of the back and front regions during normal use of the article, and is exposed upon tearing open at least a part of the side joint region.

According to one embodiment, said side joint region comprises regions outside said fastening means in which the front and back regions are joined to each other by adhesive, ultrasonic welding, heat sealing or the like, and that in other regions of the side joint region overlying said fastening means the front and back regions are substantially unattached to each other except for a possible fastening effect provided by the fastening means in cooperation with the material layer in the side joint region overlying the fastening means.

According to one aspect, said side joint region comprises at least one seam region outside said fastening means as seen in the longitudinal direction of the side joint region adjacent the waist opening and/or the leg opening in which the front and back regions are joined to each other with such a strong bonding effect that the front and/or back region materials will rupture along said at least one seam region upon tearing open said side joint region rather than a delamination of the material layers in the side joint region will occur.

In a further embodiment, the at least one seam region has a rounded or chamfered inner edge forming a tear indication along which the material layers of the front and/or back regions will rupture upon tearing open said side joint region.

According to still a further embodiment, said at least one seam region is perforated along the periphery of said rounded or chamfered edge.

According to a further aspect, said side joint region comprises at least two seam regions in which the front and back regions are joined to each other, said seam regions being located at opposite ends of the side joint region adjacent the waist opening and the respective leg opening, wherein said fastening means is located between said at least two seam regions.

It is preferred that there is a gap of at least 1 mm between the fastening means and the respective seam region.

In one aspect, said side joint region comprises a further seam region located inside said fastening means on the side thereof facing the wearer.

According to one embodiment said further seam region shows such a weak bonding effect that upon tearing open said further seam region the front and back regions will delaminate from each other.

In one aspect, the side joint region has a broadened mid region inside the fastening means on the side thereof facing the wearer so as to form a narrowing opening in the hip region of the pant diaper.

According to a further embodiment, the front and back regions are joined to each other in said side joint region with such a weak bonding effect that upon tearing open said side joint region the front and back regions will delaminate from each other and thus expose the fastening means.

### DESCRIPTION OF DRAWINGS

The invention will in the following be closer described with reference to some embodiments shown in the accompanying drawings.

FIG. 1 shows a simplified plan view of a composite material web intended to form a plurality of pant diapers.

FIG. 2 is a simplified perspective view of a pant diaper according to a first embodiment of the invention.

FIG. 3 is a view corresponding to FIG. 2 but showing the pant diaper after one of the side joints has been torn open.

FIG. 4 shows the side joint of a pant diaper according to a modified embodiment.

FIG. 5 is a simplified perspective view of a pant diaper according to a second embodiment of the invention.

FIG. 6 is a view corresponding to FIG. 5 but showing the pant diaper after one of the side joint regions has been torn open.

FIG. 7 is a perspective view of a pant diaper rolled up for disposal.

### DEFINITIONS

Absorbent Article and Pant Type Absorbent Article

The term “absorbent article” refers to products that are placed against the skin of the wearer to absorb and contain body exudates, like urine, feces and menstrual fluid. The invention mainly refers to disposable absorbent articles, which means articles that are not intended to be laundered or otherwise restored or reused as an absorbent article after use. According to the invention pant type absorbent articles are referred to, which have a defined waist opening and a pair of leg openings and which are pulled onto the body of the wearer by inserting the legs into the leg openings and pulling the article up over the waist. Examples of such pant type absorbent articles are pant diapers, sanitary pants and incontinence pants worn by incontinent adults. In the following the article will be referred to as a pant diaper.

Topsheet

The term “topsheet” refers to the liquid permeable material sheet forming the inner cover of the absorbent article and which in use is placed in direct contact with the skin of the wearer. The topsheet can comprise a nonwoven material, e g spunbond, meltblown, carded, hydroentangled, wetlaid etc. Suitable nonwoven materials can be composed of natural fibers, such as woodpulp or cotton fibres, manmade fibres, such as polyester, polyethylene, polypropylene, viscose etc. or from a mixture of natural and manmade fibres. The topsheet material may further be composed of tow fibres, which may be bonded to each other in a bonding pattern, as e.g. disclosed in EP-A-1 035 818. Further examples of topsheet materials are porous foams, apertured plastic films etc. The materials suited as topsheet materials should be soft and non-irritating to the skin and be readily penetrated by body fluid, e g urine or menstrual fluid. The topsheet may further be different in different parts of the absorbent article.

Backsheet

The term backsheet refers to the liquid impervious material forming the outer cover of the absorbent article at least on the core area thereof. The backsheet can comprise a thin plastic film, e.g. a polyethylene or polypropylene film, a nonwoven material coated with a liquid impervious material, a hydrophobic nonwoven material, which resists liquid penetration, or a laminate of a plastic film and a nonwoven material. The backsheet material may be breathable so as to allow vapour to escape from the absorbent core, while still preventing liquids from passing therethrough. Examples of breathable backsheet materials are porous polymeric films, nonwoven laminates from spunbond and meltblown layers, laminates from porous polymeric films and nonwoven materials.

Absorbent Core

The “absorbent core” is the absorbent structure disposed between the two covers of the absorbent article. The absorbent core can be of any conventional kind. Examples of commonly occurring absorbent materials are cellulosic fluff pulp, tissue layers, highly absorbent polymers (so called superabsorbents), absorbent foam materials, absorbent nonwoven materials or the like. It is common to combine cellulosic fluff pulp with superabsorbent polymers in an absorbent core. Superabsorbent polymers are water-swellable, water-insoluble organic or inorganic materials capable of absorbing at least about 20 times its weight and in an aqueous solution containing 0.9 weight percent of sodium chloride. Organic materials suitable for use as a superabsorbent material can include natural materials such as polysaccharides, polypeptides and the like, as well as synthetic materials such as synthetic hydrogel polymers. Such hydrogel polymers include, for example, alkali metal salts of polyacrylic acids, polyacrylamides, polyvinyl alcohol, polyacrylates, polyacrylamides, polyvinyl pyridines, and the like. Other suitable polymers include hydrolyzed acrylonitrile grafted starch, acrylic acid grafted starch, and isobutylene maleic anhydride copolymers and mixtures thereof. The hydrogel polymers are preferably lightly crosslinked to render the material substantially water insoluble. Preferred superabsorbent materials are further surface crosslinked so that the outer surface or shell of the superabsorbent particle, fiber, flake, sphere, etc. possesses a higher crosslink density than the inner portion of the superabsorbent. The superabsorbent materials may be in any form suitable for use in absorbent composites including particles, fibers, flakes, spheres, and the like.

A high absorption capacity is provided by the use of high amounts of superabsorbent material. For an absorbent core comprising a matrix of hydrophilic fibers, such as cellulosic fibers, and superabsorbent material, the proportion of superabsorbent material is preferably between 10 and 90% by weight, more preferably between 30 and 70% by weight.

It is conventional in absorbent articles to have absorbent cores comprising layers of different properties with respect to liquid receiving capacity, liquid distribution capacity and storage capacity. The thin absorbent bodies, which are common in for example baby diapers and incontinence guards, often comprise a compressed mixed or layered structure of cellulosic fluff pulp and superabsorbent polymers. The size and absorbent capacity of the absorbent core may be varied to be suited for different uses such as for infants or for adult incontinent persons.

The absorbent core may further include an acquisition distribution layer placed on top of the primary absorbent body and which is adapted to quickly receive and temporarily store discharged liquid before it is absorbed by the primary absorbent core. Such acquisition distribution layers are well known in the art and may be composed of porous fibrous waddings or foam materials.

Front Region

The front region of a pant-type absorbent article is defined as the part of the article that in use is intended to extend over the stomach and front hip region of the wearer.

Back Region

The back region of a pant-type absorbent article is defined as the part of the article that in use is intended to extend over the back and the rear hip region of the wearer.

Crotch Region

The crotch region of a pant-type absorbent article is defined as the part of the article that in use is intended to extend through the wearer's crotch region, between the legs.

Side Joint Region

The term “side joint regions” as used herein refers to the regions in which the lateral side edges of the front and back regions are joined to each other to form side seams, wherein the article assumes a pant-like shape having a defined waist opening and two leg openings.

Disposal Means

The term “disposal means” refers to a fastening means such as a strip of an adhesive tape or a mechanical fastening means in the form of hook members, which is arranged to keep the diaper in the folded or rolled up state after disposal thereof.

### DESCRIPTION OF PREFERRED EMBODIMENTS

The pant diaper 1 disclosed in the drawings typically comprises a front region 2, a back region 3 and a crotch region 4 between the front region and back region. In it's most common form, the pant diaper comprises a liquid pervious topsheet 5, a liquid impervious backsheet 6 and an absorbent core 7 disposed between the topsheet and the backsheet. The pant diaper is intended to enclose the lower part of the wearer's trunk like a pair of absorbent pants. The front and back regions 2 and 3 may have different material compositions than the crotch region if or when a core package is joined to the front and back regions.

The front and back regions 2 and 3 are joined to each other along two opposite side edges 2a and 3a thereof, to define a waist-opening 8 and a pair of leg-openings 9. The front and back regions 2 and 3 are joined along said side edges 2a and 3a, for example by adhesive, ultrasonic welding, heat sealing or the like, so as to form side joint regions 10. The front and back regions 2 and 3 can either be joined along their side edges 2a and 3a with the topsheet facing inwards in the side seams, as is shown in the drawings. Alternatively they are joined in an overlapped manner with the topsheet of either the front or back region facing the backsheet of the opposite region.

In an alternative form the pant diaper comprises a core region having a core package, a liquid pervious topsheet and a liquid impervious backsheet as disclosed above, and a chassis region outside the core region, wherein the chassis region comprises a coversheet of a soft and comfortable material, for example an elastic laminate, to which the core package is joined.

The waist area, at least a part of the leg opening area and the side areas adjacent the side joint regions 10 are elasticized. The elastification is usually accomplished by a plurality of elastic members, such as elastic threads 11, contractably affixed between the backsheet 6 and the topsheet 5. Alternatively elastic materials, such as an elastic laminate, may be used to form the chassis in those areas where elasticity is desired.

The article has a longitudinal direction y and a transverse direction x.

Hidden in one or both of the side joint regions 10 there is provided a fastening means 12 serving as disposal means. The disposal means 12 may be an adhesive tape strip or a strip of hook material adapted to interact with the backsheet 3 forming a fastening surface for the disposal means 12. In case the outer surface of the backsheet is a plastic film an adhesive tape strip is used as disposal means, while in the case the outer surface of the backsheet is a fibrous nonwoven material the disposal means 12 is a hook material, while the fibrous nonwoven material will act as a loop material interacting with the hooks of the disposal means. It is preferred that the material layer in the side joint region which is in close contact with the fastening surface of the disposal means interacts with the disposal means at least to some extent to provide a weak bonding effect between the two surfaces.

Suitable dimensions of the disposal means are between 1.5-5 cm, preferably 2-4 cm in length, y direction, and between 0.5-2.5 cm, preferably 0.7-1.5 cm in width, x direction.

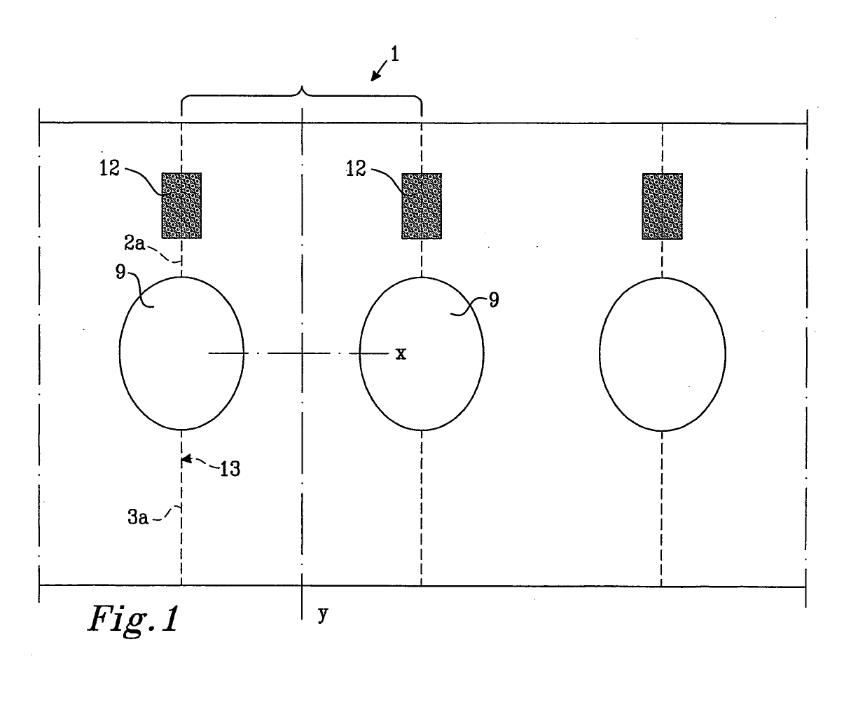

FIG. 1 shows schematically a plan view of a composite material web intended to form a plurality of pant diapers 1. Holes are cut out in the web to form leg openings 9. Disposal means 12 are attached by adhesive, ultrasonic welding, heat sealing or the like over the intended lines of separation 13 between two adjacent pant diaper blanks at the front or back region thereof. The pant diaper is formed by cutting along the separation lines 13, at which each disposal means 12 is divided in two halves, each of which forming a disposal means for a pant diaper. The side edges 2a of the front region are then joined to the side edges 3a of the rear region in the manner disclosed above, to form side joint regions 10 in which the disposal means 12 are hidden.

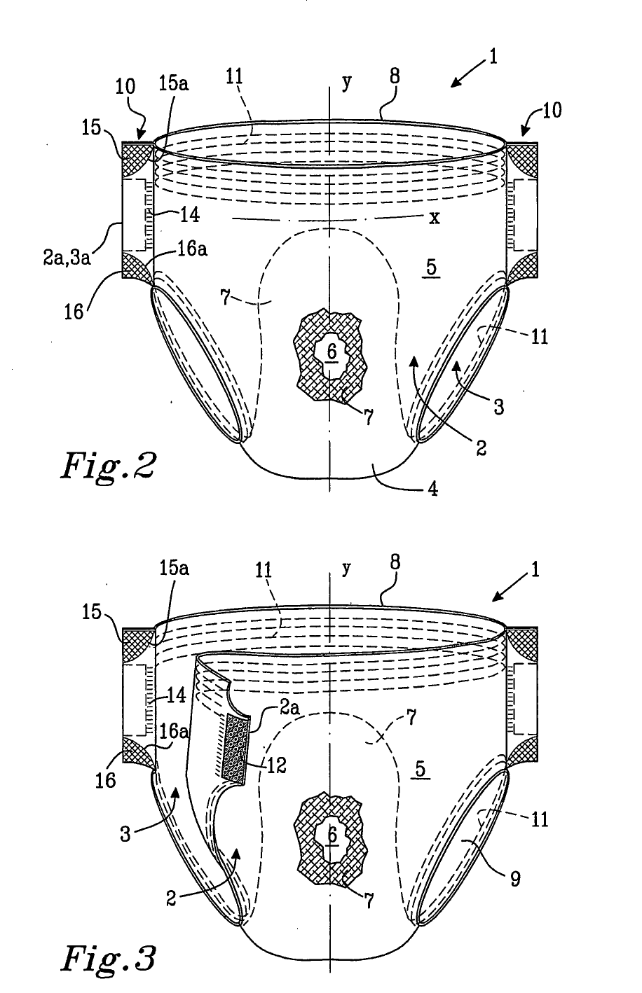

In the embodiment shown in FIGS. 2 and 3 each side joint region 10 is formed by three seam regions 14, 15 and 16 formed by adhesive, ultrasonic welding, heat sealing or the like. The first seam region 14 extends inside the disposal means 12 along at least a substantial length thereof and is relatively weak so that the seam will delaminate upon tearing open the side joint region. One important function of this first seam region 14 is to avoid a direct contact and abrasion from the disposal means against the skin of the wearer.

The second and third seam regions 15 and 16 are located at the outer corners of the side joint regions 10 adjacent the waist opening 8 and the respective leg opening 9. These seam regions 15 and 16 are relatively strong and sufficiently strong not to delaminate during normal use of the pant diaper. This usually also means that they are so strong that no delamination of the material layers in this part of the side joint region will occur upon tearing open the side joint region, but one or both materials forming either the front region 2 or back region 3 will instead rupture along the inner edges 15a and 16a of the respective seam region 15 and 16. This is illustrated in FIG. 3 of the drawings, wherein the material in the front region 5 side joint region has ruptured along the edges 15a and 16a and the front seam regions 15 and 16 remain attached to the respective back seam regions 15 and 16.

The inner edges 15a and 16a of the second and third seam regions 15 and 16 are preferably rounded or chamfered so that they will form tear indications along which the side seam will rupture upon tearing. There should preferably be a small gap between the seam regions 15 and 16 and the disposal means 12, preferably a gap of at least 1 mm. It is important that the seam regions 15 and 16 do not extend over the disposal means 12. It is herewith ensured that the side joint regions 10 may be torn open in a controlled manner and avoid that the material layers on top of and under the disposal means will rupture. The disposal means 12 may herewith be exposed and function as intended. An additional function of the disposal means 12 may be to act as a closure means in case the pant diaper has been opened along a side joint region 10 unintentionally or intentionally for checking whether the pant diaper needs to be changed or not. This reclosing function is however not mandatory and the main function of the disposal means 12 is to secure the article in a configuration that provides a convenient disposal after said article has been used.

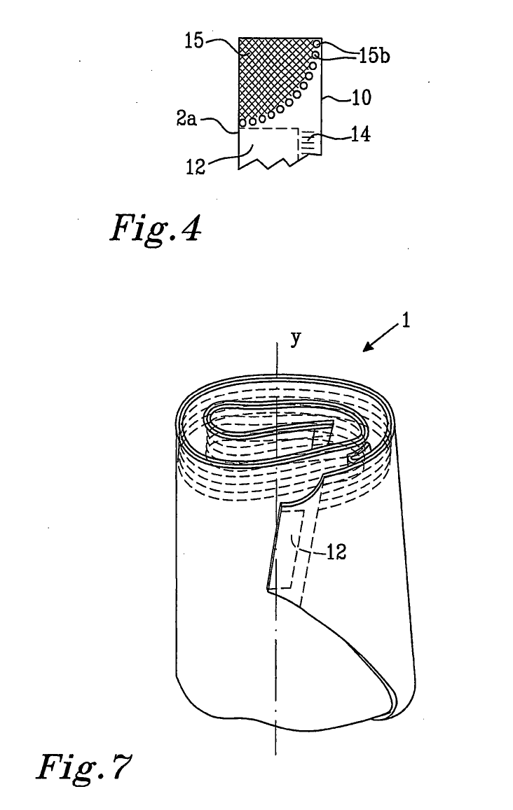

FIG. 4 shows a modified embodiment in which the seam regions 15 and 16 are perforated 15b, 16b along the periphery of the rounded or chamfered edges. This perforation 15b, 16b may be provided by a strong spotwise ultrasonic welding or heat sealing which weakens or perforates the material in one or more of the material layers forming the side joint region. The perforation act as tear indications for rupturing the side seam.

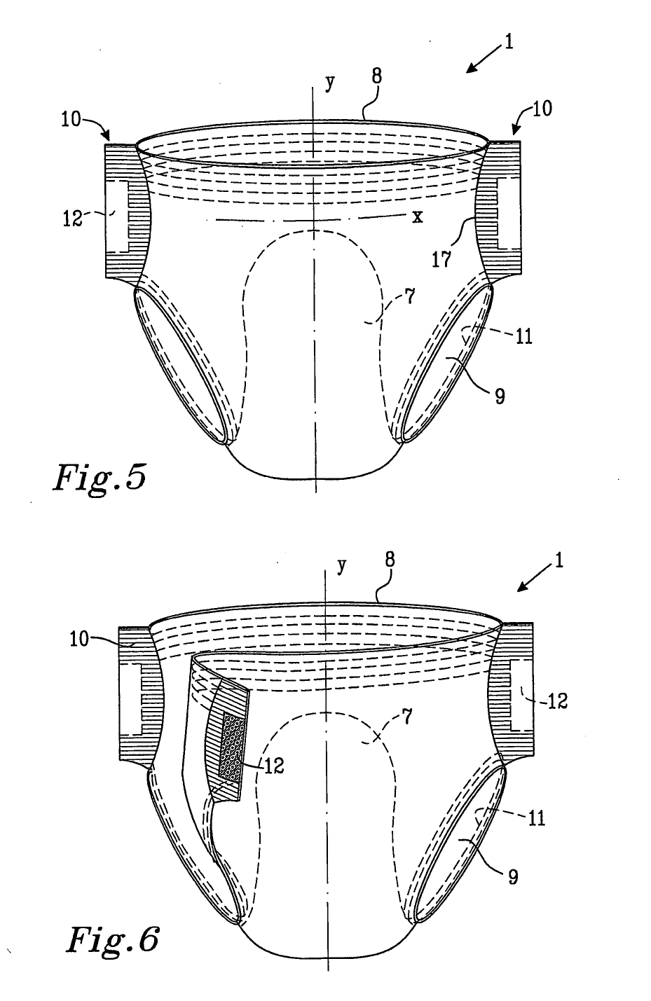

In the embodiment shown in FIGS. 5 and 6 the side joint regions 10 in their mid portion 17 are broadened and curved inwardly so as to form a narrowing opening in the hip region of the pant diaper. This means that the pant diaper will fit more tightly against the hips of the wearer and avoid that the pant diaper unintentionally will slip down. The relatively large surface area of the joined portions of the side joint region 10 means that the bonding strength per surface area of the seam does not need to be as strong as for the embodiment shown in FIGS. 2 and 3, where the seam regions are considerably smaller. The side joint region 10 is preferably weak enough to allow delamination when torn open. However it should of course be strong enough not to delaminate during normal use. A suitable bonding strength is in the range 2 to 5 N/25 mm.

FIG. 7 illustrates the pant diaper after it has been removed from the wearer after use and is rolled up and closed in this position by the disposal means 12, so that the soiled portion is wrapped inside for disposal.

The invention is of course not limited to the embodiments described above and shown in the drawings, but may be varied within the scope of the claims, and equivalents thereof. Details from the different embodiments may of course be combined as desired, for example the broadened mid portion 17 of the side joint region 10 may of course also be provided in the embodiment shown in FIGS. 2 and 3.

1. A pant type absorbent article, comprising:
a front region, a back region, and a crotch region between the front region and the back region, an absorbent core disposed at least in said crotch region, the front and back regions being joined to each other along two opposite side edges of said article to define a waist-opening and a pair of leg-openings, the front and back regions being joined along said side edges so as to form a side joint region, said article further comprising at least one fastener joined to at least one of the front and back regions for allowing the article to be secured in a configuration that provides a convenient disposal after said article has been used, said article having a longitudinal and a transverse direction,
said fastener is disposed in said side joint region and is hidden between the joined side edges of the back and front regions during normal use of the article, and is exposed upon tearing open at least a part of the side joint region.

2. The absorbent article as claimed in claim 1, wherein said side joint region comprises regions outside said fastener in which the front and back regions are joined to each other by adhesive, ultrasonic welding, or heat sealing, and that in other regions of the side joint region overlying said fasteners, the front and back regions are substantially unattached to each other except for a fastening effect provided by the fastener in cooperation with the material layer in the side joint region overlying the fastener.

3. The absorbent article as claimed in claim 2, wherein said side joint region comprises at least one seam region outside said fastener as seen in the longitudinal direction of the side joint region adjacent the waist opening and/or the leg opening in which the front and back regions are joined to each other with such a strong bonding strength that the front and/or back region material will rupture along said at least one seam region upon tearing open said side joint region rather than a delamination of the material layers in the at least one seam region will occur.

4. The absorbent article as claimed in claim 3, wherein said at least one seam region has a rounded or chamfered inner edge forming a tear indication along which the material layers of the front and/or back regions will rupture upon tearing open said side joint region.

5. The absorbent article as claimed in claim 4, wherein said at least one seam region is provided with a material weakening along the periphery of said rounded or chamfered edge.

6. The absorbent article as claimed in claim 2, wherein said side joint region comprises at least two seam regions in which the front and back regions are joined to each other, said seam regions being located at opposite ends of the side joint region as seen in the longitudinal direction of the side joint region adjacent the waist opening and the respective leg opening wherein said fastener is located between said at least two seam regions.

7. The absorbent article as claimed in claim 6, wherein there is a gap of at least 1 mm between the fastener and the respective seam region.

8. The absorbent article as claimed in claim 2, wherein said side joint region comprises a further seam region located inside said fastening means on the side thereof facing the wearer.

9. The absorbent article as claimed in claim 8, wherein said further seam region has such a weak bonding effect that upon tearing open said side joint region the front and back regions will delaminate from each other.

10. The absorbent article as claimed in claim 1, wherein the side joint region has a broadened mid region inside the fastening means on the side thereof facing the wearer so as to form a narrowing opening in the hip region of the pant diaper.

11. The absorbent article as claimed in claim 1, wherein the front and back regions are joined to each other in said side joint region with such a weak bonding strength that upon tearing open said side joint region the front and back regions will delaminate from each other and thus expose the fastening means.
